(19) United States
(12) Patent Application Publication
(10) Pub. No.: US 2013/0211315 A1
(43) Pub. Date: Aug. 15, 2013

(54) Near Quadrant Glaucoma Treatment Method

(71) Applicants: Tami Aibester, Hod Hasharon (IL); Oded M. Nissan, Modiin (IL)

(72) Inventors: Tami Aibester, Hod Hasharon (IL); Oded M. Nissan, Modiin (IL)

(21) Appl. No.: 13/396,070

(22) Filed: Feb. 14, 2012

(51) Int. Cl. A61F 9/007; A61M 37/00; A61B 17/3209

(52) U.S. Cl. 604/22; 606/167

(57) ABSTRACT

A method of treating glaucoma is described. The method includes creating an incision at or near the limbus of an eye and treating glaucoma by inserting a treatment probe through the incision to act on an eye structure adjacent to or near the incision.

### BACKGROUND OF THE INVENTION

The present invention relates to a method of performing glaucoma surgery and more particularly to a surgical procedure that utilizes an incision at or near the limbus to treat the near quadrant of the eye.

Glaucoma, a group of eye diseases affecting the retina and optic nerve, is one of the leading causes of blindness worldwide. Glaucoma results when the intraocular pressure (IOP) increases to pressures above normal for prolonged periods of time. IOP can increase due to an imbalance of the production of aqueous humor and the drainage of the aqueous humor. Left untreated, an elevated IOP causes irreversible damage the optic nerve and retinal fibers resulting in a progressive, permanent loss of vision.

The eye's ciliary body epithelium constantly produces aqueous humor, the clear fluid that fills the anterior chamber of the eye (the space between the cornea and iris). The aqueous humor flows out of the anterior chamber through the uveoscleral pathways, a complex drainage system. The delicate balance between the production and drainage of aqueous humor determines the eye's IOP.

Open angle (also called chronic open angle or primary open angle) is the most common type of glaucoma. With this type, even though the anterior structures of the eye appear normal, aqueous fluid builds within the anterior chamber, causing the IOP to become elevated. Left untreated, this may result in permanent damage of the optic nerve and retina. Eye drops are generally prescribed to lower the eye pressure. In some cases, surgery is performed if the IOP cannot be adequately controlled with medical therapy.

Only about 10% of the population suffers from acute angle closure glaucoma. Acute angle closure occurs because of an abnormality of the structures in the front of the eye. In most of these cases, the space between the iris and cornea is more narrow than normal, leaving a smaller channel for the aqueous to pass through. If the flow of aqueous becomes completely blocked, the IOP rises sharply, causing a sudden angle closure attack.

Secondary glaucoma occurs as a result of another disease or problem within the eye such as: inflammation, trauma, previous surgery, diabetes, tumor, and certain medications. For this type, both the glaucoma and the underlying problem must be treated.

FIG. 1 is a diagram of the front portion of an eye that helps to explain the processes of glaucoma. In FIG. 1, representations of the lens 110, cornea 120, iris 130, ciliary bodies 140, trabecular meshwork 150, Schlemm's canal 160, limbus 170, and sclera 180 are pictured. Anatomically, the anterior chamber of the eye includes the structures that cause glaucoma. Aqueous fluid is produced by the ciliary bodies 140 that lie beneath the iris 130 and adjacent to the lens 110 in the anterior chamber. This aqueous humor washes over the lens 110 and iris 130 and flows to the drainage system located in the angle of the anterior chamber. The angle of the anterior chamber, which extends circumferentially around the eye, contains structures that allow the aqueous humor to drain. The first structure, and the one most commonly implicated in glaucoma, is the trabecular meshwork 150. The trabecular meshwork 150 extends circumferentially around the anterior chamber in the angle. The trabecular meshwork 150 seems to act as a filter, limiting the outflow of aqueous humor and providing a back pressure producing the IOP. Schlemm's canal 160 is located beyond the trabecular meshwork 150. Schlemm's canal 160 has collector channels that allow aqueous humor to flow out of the anterior chamber. The two arrows in the anterior chamber of FIG. 1 show the flow of aqueous humor from the ciliary bodies 140, over the lens 110, over the iris 130, through the trabecular meshwork 150, and into Schlemm's canal 160 and its collector channels. The limbus 170 is the location at which the cornea 120 and sclera 180 meet. Generally, the trabecular meshwork 150 and Schlemm's canal 160 are located very close to the limbus.

As shown is FIG. 2, currently available ab-interno glaucoma treatments generally target the portion of the eye opposite the incision location 210. An ab-interno glaucoma treatment is generally one in which the trabecular meshwork 150 is treated from inside the anterior chamber of the eye (as opposed to an eb-externo method such as trabeculectomy which treats the trabecular meshwork from an incision in the sclera or outside the eye). For example, several companies have developed shunts, stents, or treatment probes 200 that are introduced through a corneal incision 210 and advanced across the anterior chamber of the eye, as shown in FIG. 2. Because of the location of the lens 110 and iris 130, the corneal incision 210 in such treatments is well anterior of the limbus 170. The location of the corneal incision 210 is such that the treatment probe 200, when advanced across the anterior chamber, does not touch the lens 110 or iris 130. If such treatment probe 200 were to touch the lens 110, a cataract may result. In addition, the treatment probe 200 may damage the iris 130 or other delicate eye structures. In order to prevent such damage, the corneal incision 210 must be located such that the angle of approach to the trabecular meshwork 150 opposite the corneal incision 210 allows for clearance of the lens 110 and iris 130. However, locating the corneal incision 210 too far anterior the limbus 170 is not ideal at least because this location is not suitable for cataract surgery (which incision is located near the limbus). Accordingly, it would be desirable to have an ab-interno surgical method for treating glaucoma that utilizes an incision at or near the limbus 170.

### SUMMARY OF THE INVENTION

In one embodiment consistent with the principles of the present invention, the present invention is a method of treating glaucoma comprising: creating an incision at or near the limbus of an eye; and treating glaucoma by inserting a treatment probe through the incision to act on an eye structure adjacent to or near the incision. The eye structure may be acted on by energy, a shunt or stent, a cutting mechanism, a manipulator, or a pharmaceutical or compound. In addition, a phacoemulsification procedure may be performed through the same incision.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a diagram of the front portion of an eye.

FIG. 2 a diagram of a prior at method ab-interno treatment method for glaucoma.

FIG. 3 is a diagram of an ab-interno glaucoma treatment method according to the principles of the present invention.

FIG. 4 is a diagram of an ab-interno glaucoma treatment method according to the principles of the present invention.

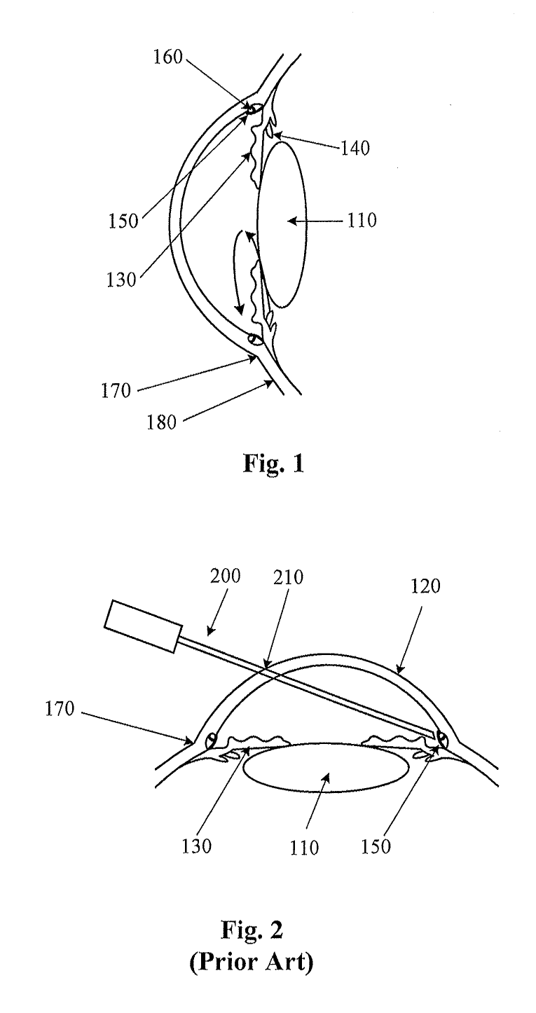

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

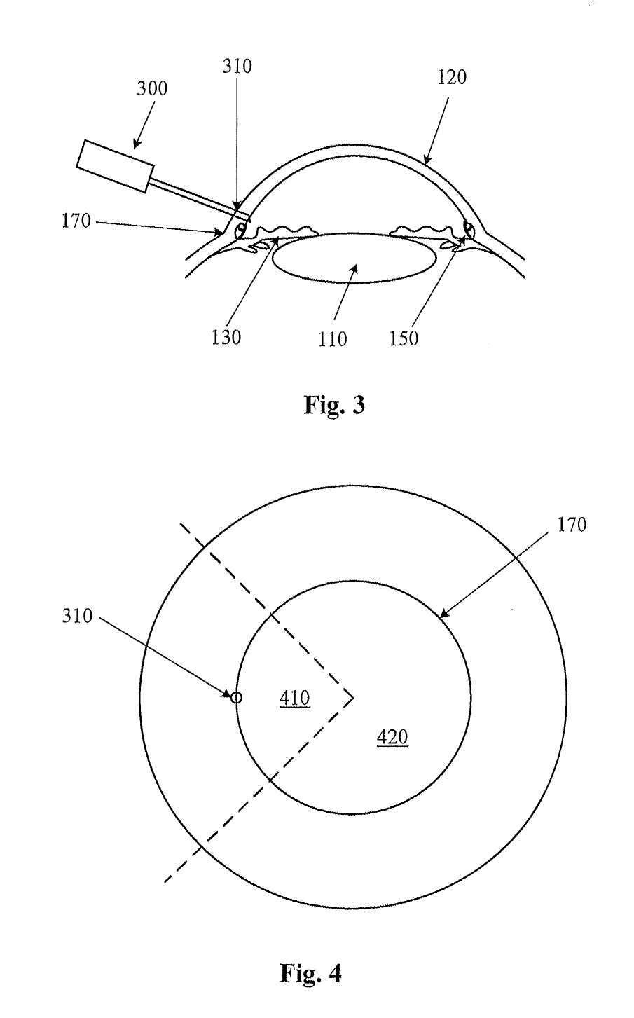

FIG. 3 is a diagram of an ab-interno glaucoma treatment method according to the principles of the present invention. In the method of FIG. 3, the incision 310 is located at or near the limbus 170. In this manner, an incision 310 is made at or near the limbus 170, and a treatment probe 300 is inserted through the incision 310 to treat the near quadrant of the eye (as described more precisely with respect to FIG. 4). Such an incision 310 may be made using a blade typically used for phacoemulsification procedures and may be of a length on the order of one or two millimeters or less. As such, the incision 310 may be self-sealing. Treatment probe 300 may be any of a number of different types of probes including but not limited to the following:
1) A probe that is designed to insert a stent or shunt through the trabecular meshwork 150 or other nearby eye structure. This type of probe may be an insertion tool that carries a shunt or stent. In practice, the end of the probe 300 is inserted through incision 310, and the shunt or stent is deposited into the eye (or through an eye structure) near the incision site 310. Because the incision site 310 is very close the trabecular meshwork 150 (and related structures such as Schlemm's canal), the probe 300 does not have to be inserted very far into the anterior chamber to place a shunt or stent. This limited travel of the probe 300 in the anterior chamber reduces the risk of damaging delicate eye structures such as the lens 110 or iris 130.
2) A probe that is designed to treat the trabecular meshwork 150 or other nearby eye structure with energy. In practice, the end of the probe 300 is inserted through incision 310, and energy is applied to an eye structure (such as the trabecular meshwork 150) near the incision site 310. Because the incision site 310 is very close the trabecular meshwork 150 (and related structures such as Schlemm's canal), the probe 300 does not have to be inserted very far into the anterior chamber to treat the eye. This limited travel of the probe 300 in the anterior chamber reduces the risk of damaging delicate eye structures such as the lens 110 or iris 130.
3) A probe that is designed to treat the trabecular meshwork 150 or other nearby eye structure with a blade or cutting mechanism. In practice, the end of the probe 300 is inserted through incision 310, and the blade or cutting mechanism is applied to an eye structure (such as the trabecular meshwork 150) near the incision site 310. Because the incision site 310 is very close the trabecular meshwork 150 (and related structures such as Schlemm's canal), the probe 300 does not have to be inserted very far into the anterior chamber to treat the eye. This limited travel of the probe 300 in the anterior chamber reduces the risk of damaging delicate eye structures such as the lens 110 or iris 130.
4) A probe that is designed to treat the trabecular meshwork 150 or other nearby eye structure with a pharmaceutical or other compound. In practice, the end of the probe 300 is inserted through incision 310, and the pharmaceutical or other compound is applied to an eye structure (such as the trabecular meshwork 150) near the incision site 310. Because the incision site 310 is very close the trabecular meshwork 150 (and related structures such as Schlemm's canal), the probe 300 does not have to be inserted very far into the anterior chamber to treat the eye. This limited travel of the probe 300 in the anterior chamber reduces the risk of damaging delicate eye structures such as the lens 110 or iris 130.
5) Other type of probe the treats or manipulates an eye structure. In practice, the end of the probe 300 is inserted through incision 310, and the eye structure (such as the trabecular meshwork 150) near the incision site 310 is treated or manipulated. Because the incision site 310 is very close the trabecular meshwork 150 (and related structures such as Schlemm's canal), the probe 300 does not have to be inserted very far into the anterior chamber to treat the eye. This limited travel of the probe 300 in the anterior chamber reduces the risk of damaging delicate eye structures such as the lens 110 or iris 130.
In addition, the location of incision 310 is suitable for the performance of cataract surgery. In one example of the present invention, incision 310 is made near the limbus of the eye, cataract surgery is performed via phacoemulsification through the incision 310, and glaucoma treatment is performed through the same incision 310.

FIG. 4 is a diagram of an ab-interno glaucoma treatment method according to the principles of the present invention. FIG. 4 shows the incision 310 located at or near the limbus 170. The dashed lines indicate the quadrant 410 that is treated through incision 310. In this manner, the near quadrant 410 (i.e. the quadrant closest to the incision 310) is treated for glaucoma. The remaining three quadrants 420 are not directly treated. The treatment of the near quadrant adjacent to the incision 310 allows delicate eye structures to be avoided. In addition, the location of the incision 310 near the treatment site allows better visualization of the treatment area. A probe can be inserted through incision 310 a very short distance to treat the area adjacent to or near the incision 310. The method of the present invention may be used to treat glaucoma by treating any of a number of different eye structures including but not limited to: the trabecular meshwork, Schlemm's canal, collector channels in Schlemm's canal, the ciliary bodies, the ciliary muscles, and the choroid. If a shunt or stent is used, the shunt or stent may bridge the trabecular meshwork. The shunt or stent may reside wholly or partially within Schlemm's canal. The shunt or stent may have an inlet end in the anterior chamber and an outlet end in Schlemm's canal, a collector channel, an episcleral vein, a lymph duct, or the choroid.

From the above, it may be appreciated that the present invention provides a method for treating glaucoma. The present invention provides a surgical procedure that utilizes an incision at or near the limbus to treat the near quadrant of the eye. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating glaucoma, the method comprising:
creating an incision at or near the limbus of an eye; and
treating glaucoma by inserting a treatment probe through the incision to act on an eye structure adjacent to or near the incision.

2. The method of claim 1 further comprising:
performing a phacoemulsification procedure through the incision.

3. The method of claim 1 further comprising:
visualizing a treatment area adjacent to or near the incision.

4. The method of claim 1 wherein inserting a treatment probe through the incision to act on an eye structure adjacent to or near the incision further comprises:
treating the trabecular meshwork or Schlemm's canal.

5. The method of claim 1 wherein inserting a treatment probe through the incision to act on an eye structure adjacent to or near the incision further comprises:
inserting a shunt or stent through the incision.

6. The method of claim 1 wherein inserting a treatment probe through the incision to act on an eye structure adjacent to or near the incision further comprises:
treating the eye structure with energy.

7. The method of claim 1 wherein inserting a treatment probe through the incision to act on an eye structure adjacent to or near the incision further comprises:
cutting the eye structure.

8. The method of claim 1 wherein inserting a treatment probe through the incision to act on an eye structure adjacent to or near the incision further comprises:
manipulating the eye structure.

9. The method of claim 1 wherein inserting a treatment probe through the incision to act on an eye structure adjacent to or near the incision further comprises:
treating the eye structure with a pharmaceutical or compound.

10. The method of claim 1 wherein inserting a treatment probe through the incision to act on an eye structure adjacent to or near the incision further comprises:
avoiding contact with a lens and an iris.

11. The method of claim 1 wherein creating an incision at or near the limbus of an eye further comprises:
creating an incision no longer than two millimeters.
